(19) United States
(12) Patent Application Publication
(10) Pub. No.: US 2026/0076609 A1
(43) Pub. Date: Mar. 19, 2026

(54) SYMPATHETIC NERVOUS SIGNAL SENSING SYSTEM AND METHOD

(71) Applicant: NATIONAL TSING HUA UNIVERSITY, Hsinchu (TW)

(72) Inventor: TING-WEI WANG, Hsinchu (TW)

(73) Assignee: NATIONAL TSING HUA UNIVERSITY, Hsinchu (TW)

(21) Appl. No.: 19/333,025

(22) Filed: Sep. 18, 2025

(30) Foreign Application Priority Data
Sep. 18, 2024 (TW) 113135229

(51) Int. Cl. A61B 5/00; A61B 5/308; A61B 5/318

(52) U.S. Cl. CPC A61B 5/4035; A61B 5/308; A61B 5/318

(57) ABSTRACT

The sympathetic nervous signal sensing system includes an electrode pair, a high input impedance receiving circuit, a differential circuit, and a post-processing circuit. The electrode pair is arranged on a target and includes a first electrode configured to receive a first signal and a second electrode configured to receive a second signal. The high input impedance receiving circuit includes a first follower configured to receive a first signal and output a third signal, and a second follower configured to receive a second signal and output a fourth signal. The differential circuit is configured to perform differential processing on the third signal and the fourth signal to generate a fifth signal. The post-processing circuit is configured to filter the fifth signal according to a first filtering frequency band to output a sympathetic nervous signal.

### TECHNICAL FIELD

The present invention relates to a sympathetic nervous signal sensing system and method; and in particular to, a non-contact sympathetic nervous signal sensing system and method.

### BACKGROUND

For monitoring or measuring conditions such as anxiety or emotional stress, sympathetic nervous activity (SNA) signals are an important indicator to be considered. In clinical applications, microneurography is a standard procedure for direct measurement of SNA signals. However, since microneurography is an invasive measurement process, it is less suitable for long-term or wearable applications.

On the other hand, although non-invasive measurement processes such as heart rate variability (HRV) using electrode patches can be employed to evaluate SNA, the electrode patches still contact human skin. Contact-type electrode patches are likely causing psychological resistance for the target, especially when being used in applications such as assessments of anxiety or emotional stress. In some situations, the operator of the electrode patches uses conductive gels or other mediums to assist with the conduction of the electrode patches. Therefore, contact-type practices for sensing SNA not only increase discomfort to the target but also pose risks of hygiene issues or allergies.

In view of the deficiencies in the prior art described above, non-invasive and non-contact methods of SNA measurement will be a key focus of development in this field.

### SUMMARY

One objective of the present invention is to provide a non-contact sympathetic nervous signal sensing system and method.

In an embodiment, the present invention provides a sympathetic nervous signal sensing system. The sympathetic nervous signal sensing system includes an electrode pair, a high input impedance receiving circuit, a differential circuit, and a post-processing circuit. The electrode pair is disposed outside a sympathetic nervous signal source of a target and includes a first electrode configured to receive a first signal and a second electrode configured to receive a second signal. The high input impedance receiving circuit is coupled to the electrode pair and includes a first follower configured to receive the first signal and output a third signal approximately equal to the first signal, and a second follower configured to receive the second signal and output a fourth signal approximately equal to the second signal. The differential circuit is coupled to the high input impedance receiving circuit and is configured to perform differential processing on the third signal and the fourth signal to generate a fifth signal. The post-processing circuit is coupled to the high input impedance receiving circuit and is configured to filter the fifth signal according to a first filtering frequency band to output a sympathetic nervous signal.

In an embodiment, the present invention provides a sympathetic nervous signal sensing method. The method includes: disposing an electrode pair outside a sympathetic nervous signal source of a target, wherein the electrode pair includes a first electrode configured to receive a first signal and a second electrode configured to receive a second signal; receiving the first signal with a high input impedance receiving circuit and outputting a third signal approximately equal to the first signal, and receiving the second signal with the high input impedance receiving circuit and outputting a fourth signal approximately equal to the second signal; performing differential processing on the third signal and the fourth signal with a differential circuit to generate a fifth signal; and filtering the fifth signal with a post-processing circuit according to a first filtering frequency band to output a sympathetic nervous signal.

As described above, through the high input impedance receiving circuit, it is possible to effectively offset the equivalent impedance caused by various medium (for example, skin, muscle, air, clothing, or hair) between the sympathetic nervous signal source and the electrode pair. The followers of the high input impedance receiving circuit provide high input impedance and low output impedance, thereby enabling impedance isolation between the electrode pair and the back-end/following circuitry, and further reducing crosstalk between the electrode pair and the back-end/following circuitry.

In an embodiment, the system further includes a negative impedance circuit coupled between the electrode pair and the high input impedance receiving circuit. The negative impedance circuit is configured to provide a negative impedance value approximately equal to a negative value of the equivalent impedance from the sympathetic nervous signal source to the electrode pair to the input terminals of the first follower and the second follower.

In an embodiment, the system further includes a controller coupled between the post-processing circuit and the negative impedance circuit, wherein the controller is configured to adjust the negative impedance value of the negative impedance circuit according to at least one signal parameter of the sympathetic nervous signal.

In an embodiment, the system further includes a controller coupled between the post-processing circuit and the negative impedance circuit, wherein the post-processing circuit is further configured to filter the fifth signal according to a second filtering frequency band to output an electrocardiogram (ECG) signal; and wherein the controller is configured to adjust the negative impedance value of the negative impedance circuit according to at least one signal parameter of the ECG signal.

As described above, by providing a negative impedance value through the negative impedance circuit, the equivalent impedance difference between the sympathetic nervous signal source and the electrode pair is reduced or eliminated. Therefore, in the present invention, even when the electrode pair does not contact the skin of the target, the accuracy of the sympathetic nervous signal is further improved through impedance compensation by the negative impedance circuit. Moreover, in an embodiment, the negative impedance of the negative impedance circuit is further adjusted according to feedback from the measured signal to achieve optimal compensation.

### BRIEF DESCRIPTION OF THE DRAWINGS

The accompanying drawings are presented to help describe various aspects of the present invention. In order to simplify the accompanying drawings and highlight the contents to be presented in the accompanying drawings, conventional structures or elements in the accompanying drawings may be drawn in a simple schematic way or may be omitted. For example, a number of elements may be singular or plural. These accompanying drawings are provided merely to explain these aspects and not to limit them.

FIG. 1 illustrates a schematic diagram of impedance measurement according to the invention.

FIG. 2 illustrates a block diagram and measurement schematic of one embodiment of the system.

FIG. 3 illustrates an embodiment of the system including a backend device.

FIG. 4 illustrates an embodiment including multiple filtering frequency bands.

FIG. 5 illustrates an embodiment including bias resistors.

FIG. 6 illustrates an embodiment including an AC coupling circuit.

FIG. 7 illustrates a circuit architecture.

FIG. 8 illustrates an embodiment including a common-mode electrode.

FIG. 9 illustrates another circuit architecture.

FIG. 10 illustrates an embodiment including a negative impedance circuit.

FIG. 11 illustrates an embodiment including a controller for adjusting the negative impedance circuit.

FIG. 12 illustrates another embodiment including a controller for adjusting the negative impedance circuit.

### DETAILED DESCRIPTION

Any reference to elements using terms such as “first” and “second” herein generally does not limit the number or order of these elements. Conversely, these names are used herein as a convenient way to distinguish two or more elements or element instances. Therefore, it should be understood that the terms “first” and “second” in the request item do not necessarily correspond to the same names in the written description. Furthermore, it should be understood that references to the first element and the second element do not indicate that only two elements can be used or that the first element needs to precede the second element. Open terms such as “include”, “comprise”, “have”, “contain”, and the like used herein means including but not limit to.

The term “coupled” is used herein to refer to direct or indirect electrical coupling between two structures. For example, in an example of indirect electrical coupling, one structure may be coupled with another structure through a passive element such as a resistor, a capacitor, or an inductor.

In the present invention, the term such as “exemplary” or “for example” is used to represent “giving an example, instance, or description”. Any implementation or aspect described herein as “exemplary” or “for example” is not necessarily to be construed as preferred or advantageous over other aspects of the present invention. The terms “about” and “approximately” as used herein with respect to a specified value or characteristic are intended to represent within a value (for example, 10%) of the specified value or characteristic.

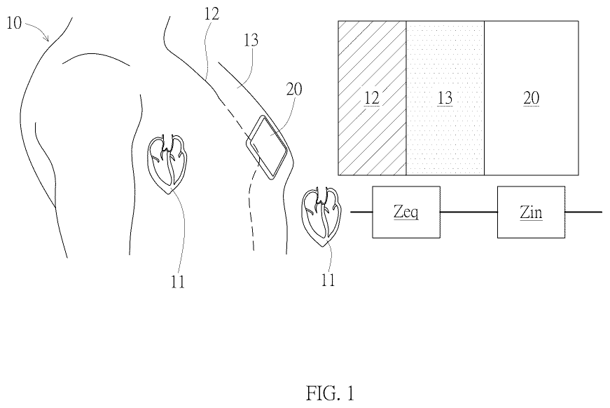

Referring to FIG. 1, various mediums (for example, skin 12 or clothing 13) exist between the sympathetic nervous signal source 11 of the target 10 (for example, the heart of the target 10) and the electrode pair 20. These mediums have different equivalent impedances Zeq depending on their conductivity or dielectric constant. Therefore, when using the electrode pair 20 to acquire sympathetic nervous-related signals of the target 10 in a non-contact manner, the acquisition of the electrode pair 20 is affected by the equivalent impedance Zeq and the input impedance Zin of the sensing device. Accordingly, the present invention provides a sympathetic nervous signal sensing system having a high input impedance Zin.

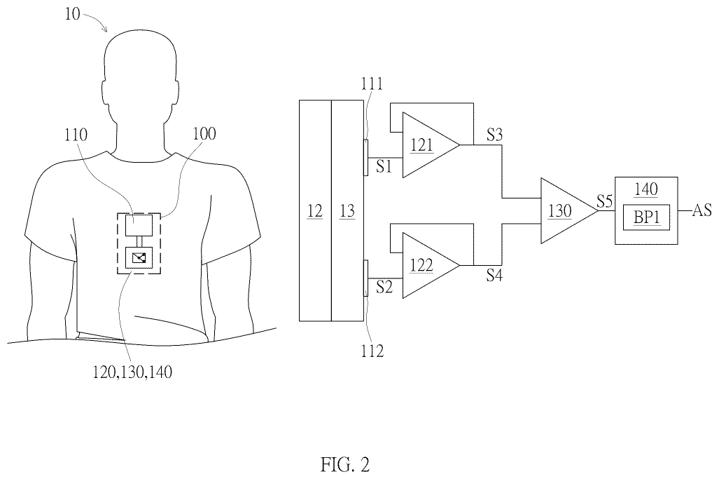

Referring to FIG. 2, FIG. 2 illustrates a sympathetic nervous signal sensing system 100 according to one embodiment of the present invention. The sympathetic nervous signal sensing system 100 includes an electrode pair 110, a high input impedance receiving circuit 120, a differential circuit 130, and a post-processing circuit 140. The electrode pair 110 is disposed outside a sympathetic nervous signal source of a target 10. In an embodiment, the sympathetic nervous signal source is sinoatrial node of the target 10, and the electrode pair 110 is disposed at the chest or back of the target. The electrode pair 110 includes a first electrode 111 configured to receive a first signal S1 and a second electrode 112 configured to receive a second signal S2. The high input impedance receiving circuit 120 is coupled to the electrode pair 110 and includes a first follower 121 configured to receive the first signal S1 and output a third signal S3 approximately equal (±1%-±20%) to the first signal S1, and a second follower 122 configured to receive the second signal S2 and output a fourth signal S4 approximately equal to the second signal S2. The differential circuit 130 is coupled to the high input impedance receiving circuit 120 and is configured to perform differential processing on the third signal S3 and the fourth signal S4 to generate a fifth signal S5. The post-processing circuit 140 is coupled to the high input impedance receiving circuit 120 and is configured to filter the fifth signal S5 according to a first filtering frequency band BP1 to output a sympathetic nervous signal AS.

In an embodiment, the electrode pair 110 is disposed outside the sympathetic nervous signal source of the target 10 through a mounting structure. For example, the mounting structure includes fastening means such as straps or hook-and-loop fasteners attached to the exterior of the target's chest or back (for example, on clothing). In an embodiment, the electrode pair 110 are disposed on or within structures such as a mattress or chair back, such that by sitting or lying, the target 10 brings the electrode pair 110 into proximity with the exterior of the sympathetic nervous signal source. The first electrode 111 and the second electrode 112 are arranged in accordance with conventional electrode configurations for measuring electrocardiogram signals. In an embodiment, the first electrode 111 and the second electrode 112 are disposed on the skin exterior, where the shortest distance to the sinoatrial node of the target's heart. It should be noted that the present invention is not limited by the type of medium between the electrode pair 110 and the skin of the target. In an embodiment, the medium between the electrode pair 110 and the skin is air, woven fabric, conductor wire fabric, or dielectric materials (for example, PDMS). In an embodiment, the first electrode 111 and the second electrode 112 are configured to be flexible capacitive electrodes. In this embodiment, the electrode pair 110 includes polymer foam wrapped with conductive textile on the outer layer. The flexible capacitive electrodes can conform to the curvature of the body, thereby significantly reducing gaps between the electrode pair 110 and the target 10, and allowing the medium state between the electrode pair 110 and the skin to remain stable.

The high input impedance receiving circuit 120 is composed of a first follower 121 and a second follower 122. The first follower 121 and the second follower 122 correspond respectively to the first electrode 111 and the second electrode 112. The first follower 121 and the second follower 122 are implemented by any components having high input impedance, such as amplifiers, transistor circuits, or instrumentation amplifiers. Preferably, the first follower 121 and the second follower 122 are configured to have high input impedance and low output impedance, thereby allowing a larger voltage drop ratio to be obtained from the electrode pair and providing the voltage to the subsequent stage circuit. Accordingly, the output signal of the first follower 121 (i.e., the third signal S3) is approximately equal to the first signal S1 from the first electrode 111, and the output signal of the second follower 122 (i.e., the fourth signal S4) is approximately equal to the second signal S2 from the second electrode 112.

In an embodiment, the differential circuit 130 is composed of components such as an instrumentation amplifier or a low-noise amplifier. The differential circuit 130 performs differential processing on the third signal S3 and the fourth signal S4 provided by the high input impedance receiving circuit 120. In this way, the difference signal between the third signal S3 and the fourth signal S4 (i.e., the fifth signal S5) is extracted and amplified.

In an embodiment, the post-processing circuit 140 is a filter circuit, or a signal processing circuit integrating a filter circuit and a signal amplification circuit. In an embodiment, the filter circuit is a band-pass filter, or an equivalent circuit composed of a high-pass filter and a low-pass filter. The filtering band of the filter circuit (i.e., the upper and lower cutoff frequencies of the band-pass filter) is selected according to the region of interest (ROI). In applications involving the sympathetic nervous system, the filtering band is selected based on the commonly recognized frequency range of the sympathetic nervous signal AS in the medical field. In an embodiment, the first filtering frequency band BP1 for the sympathetic nervous signal AS is 500-1000 Hz to avoid frequency for ECG signals and EMG signals or to prevent noise introduced above 1000 Hz. After the fifth signal S5 is filtered by the post-processing circuit 140, signal components outside the first filtering frequency band BP1 can be excluded, thereby retaining the sympathetic nervous signal AS of the target 10.

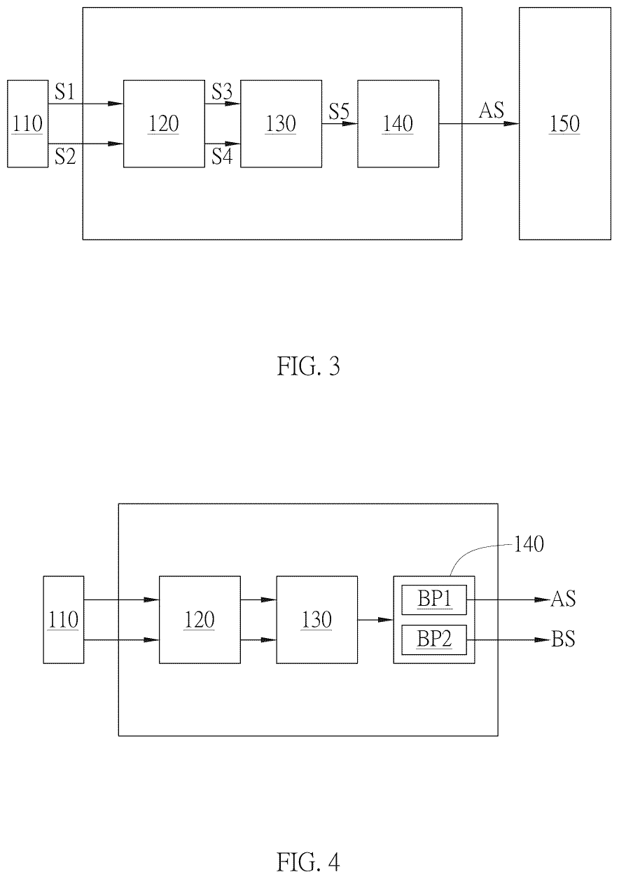

In an embodiment, the sympathetic nervous signal AS of the target 10 is transmitted to a backend device 150 via communication means. As shown in FIG. 3, the sympathetic nervous signal sensing system 100 further includes a backend device 150. The backend device 150 is configured to receive the sympathetic nervous signal AS and perform at least one signal processing operation thereon. In an embodiment, the backend device 150 is an electronic device such as a computer, tablet, or smartphone. The backend device 150 is coupled via a physical connection, such as a data acquisition (DAQ) system, to capture the sympathetic nervous signal AS from the post-processing circuit 140. In an embodiment, the post-processing circuit 140 includes an analog-to-digital conversion module or a wireless communication module to achieve wireless communication coupling with the backend device 150. In an embodiment, the backend device 150 performs signal post-processing by software (for example, a programmed instruction set) or hardware (for example, signal processing components such as an averager or integrator). In an embodiment, the at least one signal processing includes at least one of averaging, integration, accumulation, or rectification. In an embodiment, the sympathetic nervous signal AS may be rectified to obtain an absolute value signal |AS| of the sympathetic nervous signal AS. In an embodiment, the sum of the sympathetic nervous signal AS within a specific time window (for example, 100 ms) is calculated to represent an integrated sympathetic nervous signal. Finally, the integrated sympathetic nervous signal may be averaged over a time unit length of a specific number of seconds to obtain an averaged sympathetic nervous signal value. However, the post-processing method is not limited thereto.

In an embodiment, the post-processing circuit 140 has multiple filtering frequency bands for processing bioelectrical signals BS related to sympathetic nerves (for example, electromyography (EMG) signals or electrocardiogram (ECG) signals). Referring to FIG. 4, the post-processing circuit 140 is further configured to filter the fifth signal S5 according to a second filtering frequency band BP2 to output an ECG signal. The second filtering frequency band BP2 corresponding to the ECG signal is, for example, in a range of 0.05-150 Hz. It should be noted that FIG. 4 is merely an example, and the post-processing circuit 140 is able to have multiple filtering frequency bands, such as simultaneously filtering for both ECG signals and EMG signals (25-500 Hz).

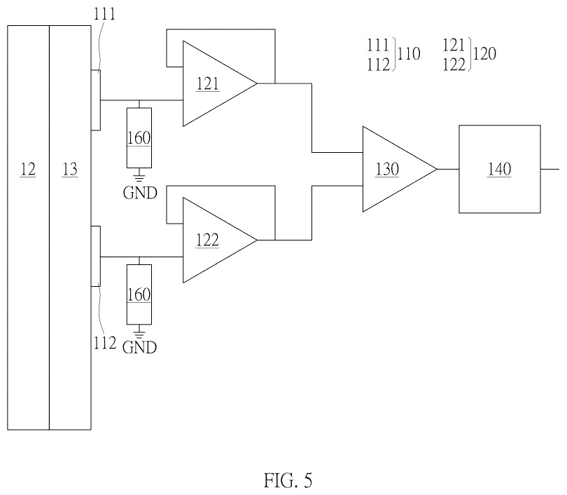

In an embodiment, the sympathetic nervous signal sensing system 100 may further include bias resistors 160. Specifically, referring to FIG. 5, the bias resistors 160 are coupled between the input terminal of the first follower 121 and the ground terminal GND, as well as between the input terminal of the second follower 122 and the ground terminal GND. The primary function of the bias resistors 160 is to discharge static charges on the clothing 13 or the skin 12 to the ground terminal GND. In an embodiment, the bias resistor 160 has a resistance value greater than 624 kΩ. The resistance value of the bias resistor 160 may be selected based on the need to eliminate static electricity while avoiding an adverse impact on the overall frequency response of the circuit. For example, the bias resistor 160 and the clothing 13 and/or the skin 12 may form an equivalent circuit of a high-pass filter, thereby influencing the measurement of the sympathetic nervous signal AS. Such influence may include, for example, high-frequency signals being introduced to the ground terminal GND via the bias resistor 160, but is not limited thereto.

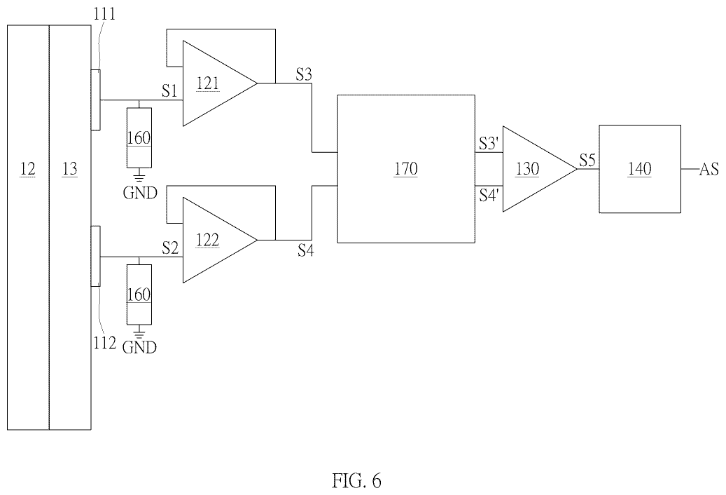

In an embodiment, the sympathetic nervous signal sensing system 100 further includes an AC coupling circuit 170. Specifically, referring to FIG. 6, the AC coupling circuit 170 is coupled between the high input impedance receiving circuit 120 and the differential circuit 130. The AC coupling circuit 170 is configured to eliminate the DC components of the third signal S3 and the fourth signal S4 while retaining their AC portions (S3′, S4′). The AC coupling circuit 170 is disposed after the high input impedance receiving circuit 120 and before the differential circuit 130. The primary function of the AC coupling circuit 170 is to remove DC offsets present in the third signal S3 and the fourth signal S4. In an embodiment, the cutoff frequency of the AC coupling circuit 170 is designed to be 0.03 Hz. In this way, during the measurement of the sympathetic nervous signal AS, interference from DC signals can be further reduced, thereby avoiding misinterpretation of cardiac rhythm signals or other signals.

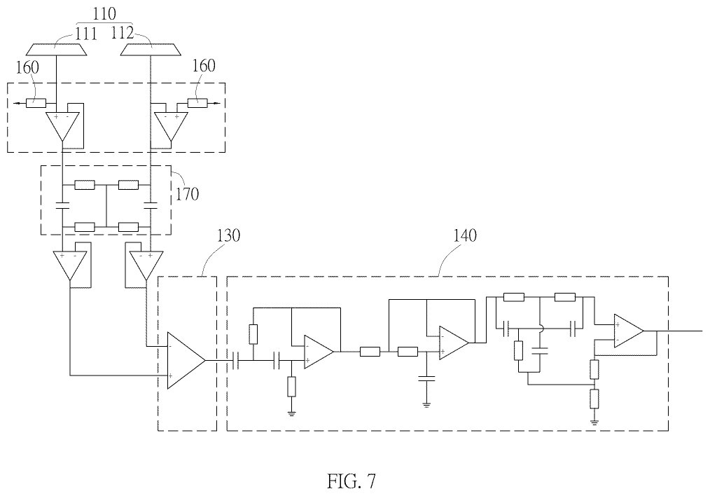

In summary of the above embodiments, the present invention proposes a specific circuit architecture. Referring to FIG. 7, the AC coupling circuit 170 includes a circuit formed of resistors and capacitors without grounding, serving as a high-pass filter to eliminate DC components. Between the AC coupling circuit 170 and the differential circuit 130, two high-impedance voltage followers may be provided as output stages to prevent signal attenuation. The post-processing circuit 140 may include a band-pass filter and amplification circuit composed of a Butterworth low-pass filter and a Butterworth high-pass filter. It should be noted that the circuit architecture shown in FIG. 7 is intended to illustrate the feasibility of implementing the present invention, and is not intended to limit the circuit architecture of the invention.

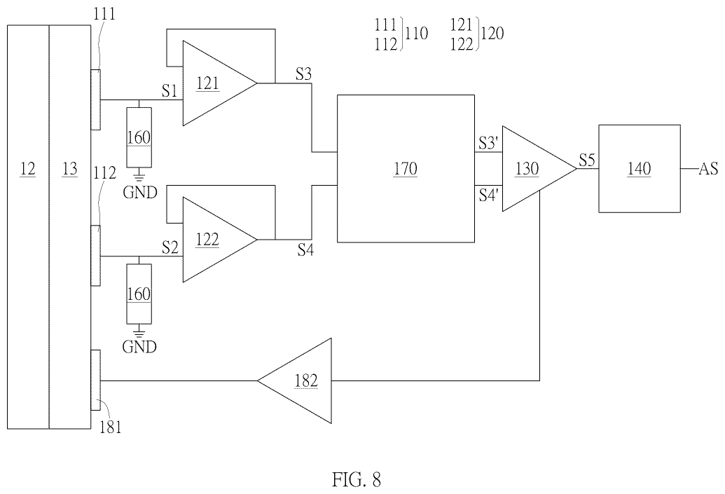

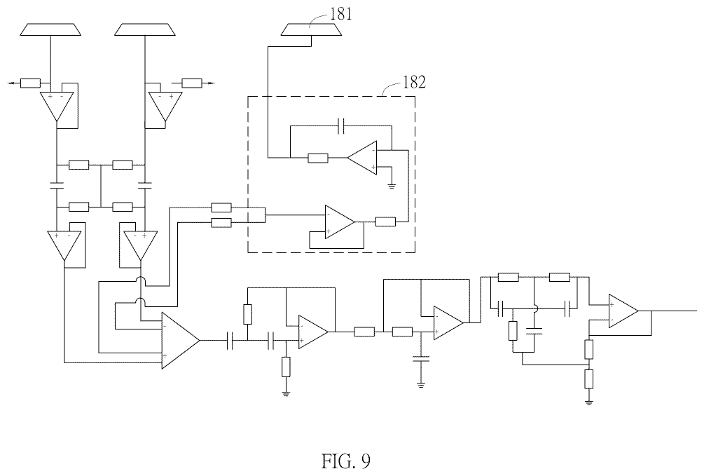

In an embodiment, the sympathetic nervous signal sensing system 100 may further include a common-mode electrode 181 and its corresponding circuit 182 (for example, a right-leg drive circuit). Specifically, referring to FIG. 8, the common-mode electrode 181 is disposed at any suitable position to receive a signal in common mode with the ground electrode and/or the second electrode 112. For example, the common-mode electrode 181 is disposed outside the sympathetic nervous signal source of the target 10 and integrated with the electrode pair to form an integrated patch. The common-mode electrode 181 is configured to receive a common-mode signal corresponding to the first signal S1. The differential circuit 130 is further configured to perform differential processing between the common-mode signal and each of the third signal S3 and the fourth signal S4. The common-mode electrode 181 and its corresponding circuit 182 are used to reduce common-mode interference in bio-signals. For example, the common-mode electrode 181 and its corresponding circuit 182 are configured to eliminate common-mode signals generated by the target 10 under the influence of external electromagnetic waves (such as environmental electromagnetic waves or power line electromagnetic interference). For the specific circuit architecture of this embodiment, referring to FIG. 9. It should be noted that the circuit architecture shown in FIG. 9 is intended to illustrate the feasibility of implementing the present invention, and is not intended to limit the circuit architecture of the invention.

As described above, the high input impedance receiving circuit 120 can effectively offset the equivalent impedance generated by various medium between the sympathetic nervous signal source and the electrode pair. The followers within the high input impedance receiving circuit 120 provide high input impedance and low output impedance, thereby enabling impedance isolation between the electrode pair and the remaining circuitry. This further reduces crosstalk between the electrode pair and the remaining circuitry. Moreover, through additional circuit configurations, such as the bias resistor 160, the AC coupling circuit 170, or the common-mode electrode 181 as described in the embodiments, the quality of signal measurement can be further improved. It should be noted that the above embodiments may be implemented individually or in combination, and the present invention is not limited to the specific embodiments.

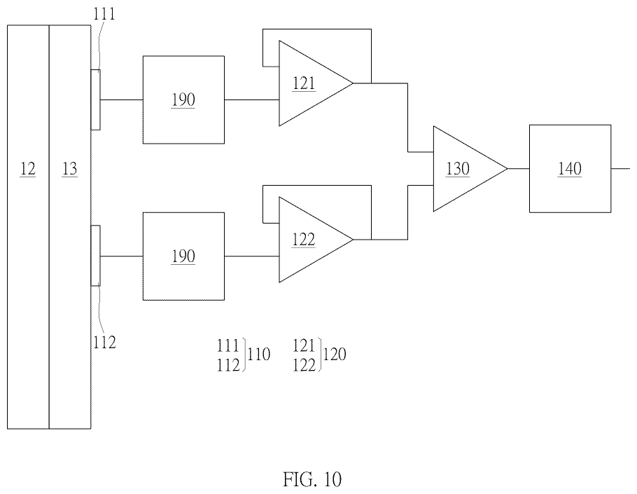

In an embodiment, the sympathetic nervous signal sensing system 100 further includes a negative impedance circuit 190. Specifically, referring to FIG. 10, the negative impedance circuit 190 is coupled between the electrode pair and the high input impedance receiving circuit 120, wherein the negative impedance circuit 190 is configured to provide a negative impedance value approximately equal to the negative value of the equivalent impedance from the sympathetic nervous signal source AS to the electrode pair to the input terminals of the first follower 121 and the second follower 122. In this embodiment, in addition to using the high input impedance receiving circuit 120 to reduce the effect of the skin or clothing on signal reception, the negative impedance circuit 190, disposed between the electrode pair and the high input impedance receiving circuit 120, can further reduce impedance mismatch problems caused by dielectric values of the skin, clothing, air, or other medium between the electrode pair and the sympathetic nervous signal source. In an embodiment, the negative impedance value of the negative impedance circuit 190 may be preset to a preset negative impedance value. For example, when the wearing environment of the target 10 is uniform (such as standardized clothing or standardized wearing distance), the preset negative impedance value can be used to reduce the operational difficulty for the operator. In an embodiment, the negative impedance value of the negative impedance circuit 190 is configured to be adjustable. For example, the negative impedance circuit 190 includes adjustable active or passive components, which are tuned via control signals or mechanical adjustment to vary the electrical characteristics of the components (such as resistance, capacitance, or inductance). By means of the adjustable negative impedance circuit 190, the signal-to-noise ratio can be optimized for different usage scenarios.

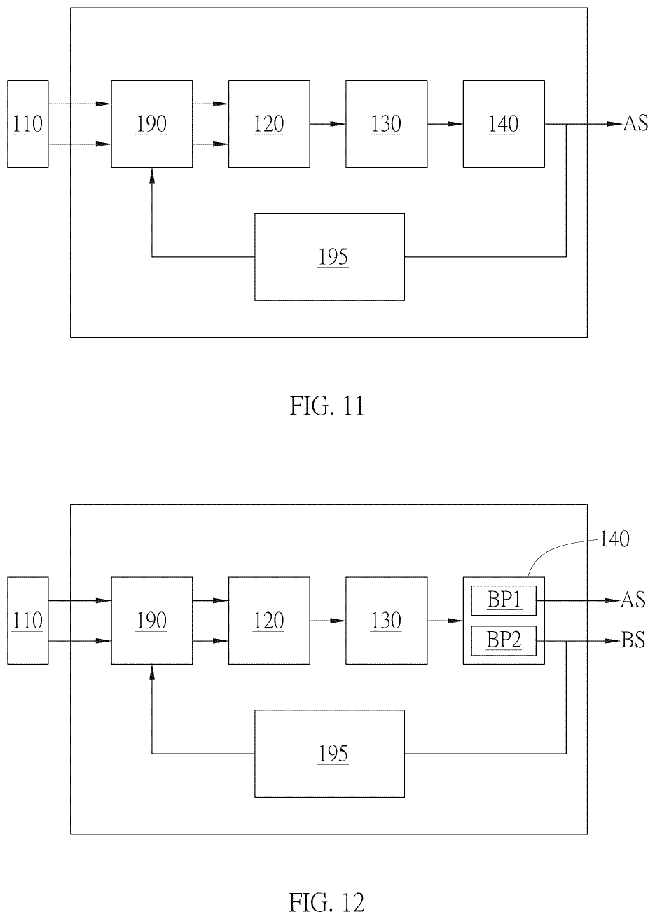

In an embodiment, the negative impedance circuit 190 is able to adjust its negative impedance value according to at least one signal parameter of the sympathetic nervous signal AS. Specifically, referring to FIG. 11, the sympathetic nervous signal sensing system 100 further includes a controller 195. The controller 195 is coupled between the post-processing circuit 140 and the negative impedance circuit 190. The controller 195 is configured to adjust the negative impedance value of the negative impedance circuit 190 based on at least one signal parameter of the sympathetic nervous signal AS. For example, the controller 195 is configured to adjust the negative impedance value of the negative impedance circuit 190 according to the measured signal parameters of the sympathetic nervous signal AS (such as amplitude, baseline level, spectrum, or signal-to-noise ratio). In one specific embodiment, the controller 195 determines whether the baseline level of the sympathetic nervous signal AS is greater than a baseline threshold (for example, defined as 30 μV). When the baseline level is lower than the baseline threshold, the controller 195 adjusts the negative impedance value of the negative impedance circuit 190, or issues an alert to enable the operator to make adjustments.

In an embodiment, the negative impedance circuit 190 is configured to be adjusted its negative impedance value according to at least one signal parameter of a bioelectrical signal related to the sympathetic nervous signal AS. For example, referring to FIG. 12, the sympathetic nervous signal sensing system 100 further includes a controller 195 coupled between the post-processing circuit 140 and the negative impedance circuit 190. The post-processing circuit 140 is further configured to filter the fifth signal S5 with a second filtering frequency band BP2 to output a bioelectrical signal (for example, an electrocardiogram (ECG) signal). The controller 195 is configured to adjust the negative impedance value of the negative impedance circuit 190 according to at least one signal parameter of the output bioelectrical signal (such as amplitude, baseline level, spectrum, or signal-to-noise ratio). Specifically, known or more distinct bioelectrical signals with higher signal-to-noise ratios are used as a basis for adjustment. For example, when various states of the ECG signal (such as PQRS waveforms) can be determined, the corresponding sympathetic nervous signal AS can also be inferred. Since both the ECG signal and the sympathetic nervous signal AS are received by the electrode pair and processed through the negative impedance circuit 190 and other circuits, the ECG signal can serve as a reference for adjustment. When the negative impedance value of the negative impedance circuit 190 is adjusted such that parameters of the ECG signal, such as signal-to-noise ratio or baseline level, reach the expected quality, the sympathetic nervous signal AS can likewise obtain improved signal quality by simultaneously adjusting the negative impedance value.

By providing a negative impedance value through the negative impedance circuit 190, the equivalent impedance difference between the sympathetic nervous signal source and the electrode pair can be reduced. Therefore, in the present invention, even when the electrode pair does not contact the skin of the target 10, the accuracy of the sympathetic nervous signal AS can be further improved through compensation by the negative impedance circuit 190. Moreover, in the embodiments described above, the negative impedance of the negative impedance circuit 190 can be further adjusted according to feedback from the measured signals to achieve optimal compensation

The aforementioned description of the present invention is provided to enable a person of ordinary skill in the art to make or implement the present invention. Various modifications to the present invention will be apparent to a person skilled in the art, and the general principles defined herein can be applied to other variations without departing from the spirit or scope of the present invention. Therefore, the present invention is not intended to be limited to the examples described herein, but is to be in accord with the widest scope consistent with the principles and novel features of the invention herein.

What is claimed is:

1. A sympathetic nervous signal sensing system, comprising:
an electrode pair disposed outside a sympathetic nervous signal source of a target, wherein the electrode pair includes:
a first electrode configured to receive a first signal; and
a second electrode configured to receive a second signal;
a high input impedance receiving circuit coupled to the electrode pair, wherein the high input impedance receiving circuit includes:
a first follower configured to receive the first signal and output a third signal approximately equal to the first signal; and
a second follower configured to receive the second signal and output a fourth signal approximately equal to the second signal;
a differential circuit coupled to the high input impedance receiving circuit, wherein the differential circuit is configured to perform a differential process on the third signal and the fourth signal to generate a fifth signal; and
a post-processing circuit coupled to the high input impedance receiving circuit, wherein the post-processing circuit is configured to filter the fifth signal with a first filtering band to output a sympathetic nervous signal.

2. The sympathetic nervous signal sensing system of claim 1, further comprising:
a common-mode electrode configured to receive a common-mode signal corresponding to the first signal; and
wherein the differential circuit is further configured to perform the differential process on the common-mode signal with the third signal and the fourth signal respectively.

3. The sympathetic nervous signal sensing system of claim 1, further comprising a backend device configured to receive the sympathetic nervous signal and perform at least one signal processing on the sympathetic nervous signal.

4. The sympathetic nervous signal sensing system of claim 1, wherein the post-processing circuit is further configured to filter the fifth signal with a second filtering band to output an electrocardiogram (ECG) signal.

5. The sympathetic nervous signal sensing system of claim 1, further comprising an AC coupling circuit coupled between the high input impedance receiving circuit and the differential circuit, wherein the AC coupling circuit is configured to remove DC components of the third signal and the fourth signal.

6. The sympathetic nervous signal sensing system of claim 1, further comprising a bias resistor coupled between an input terminal of the first follower and ground.

7. The sympathetic nervous signal sensing system of claim 1, further comprising a negative impedance circuit coupled between the electrode pair and the high input impedance receiving circuit, wherein the negative impedance circuit is configured to provide a negative impedance value approximately equal to a negative value of an equivalent impedance from the sympathetic nervous signal source to the electrode pair, to input terminals of the first follower and the second follower.

8. The sympathetic nervous signal sensing system of claim 7, further comprising a controller coupled between the post-processing circuit and the negative impedance circuit, wherein the controller is configured to adjust the negative impedance value of the negative impedance circuit based on at least one signal parameter of the sympathetic nervous signal.

9. The sympathetic nervous signal sensing system of claim 7, further comprising a controller coupled between the post-processing circuit and the negative impedance circuit, wherein:
the post-processing circuit is further configured to filter the fifth signal with a second filtering band to output an electrocardiogram (ECG) signal; and
the controller is configured to adjust the negative impedance value of the negative impedance circuit based on at least one signal parameter of the electrocardiogram signal.

10. A sympathetic nervous signal sensing method, comprising:
disposing an electrode pair outside a sympathetic nervous signal source of a target, the electrode pair comprising a first electrode configured to receive a first signal and a second electrode configured to receive a second signal;
using a high input impedance receiving circuit to receive the first signal and output a third signal approximately equal to the first signal, and to receive the second signal and output a fourth signal approximately equal to the second signal;
using a differential circuit to perform a differential process on the third signal and the fourth signal to generate a fifth signal; and
using a post-processing circuit to filter the fifth signal with a first filtering band to output a sympathetic nervous signal.

11. The sympathetic nervous signal sensing method of claim 10, further comprising:
disposing a common-mode electrode configured to receive a common-mode signal corresponding to the first signal; and
further performing the differential process on the common-mode signal with the third signal and the fourth signal using the differential circuit.

12. The sympathetic nervous signal sensing method of claim 10, further comprising using a backend device to receive the sympathetic nervous signal and perform at least one signal processing on the sympathetic nervous signal.

13. The sympathetic nervous signal sensing method of claim 10, further comprising filtering the fifth signal with a second filtering band using the post-processing circuit to output an electrocardiogram (ECG) signal.

14. The sympathetic nervous signal sensing method of claim 10, further comprising using an AC coupling circuit to remove DC components of the third signal and the fourth signal.

15. The sympathetic nervous signal sensing method of claim 10, further comprising using a negative impedance circuit to provide a negative impedance value approximately equal to a negative value of an equivalent impedance from the sympathetic nervous signal source to the electrode pair, to an input terminal of the high input impedance receiving circuit.

16. The sympathetic nervous signal sensing method of claim 15, further comprising using a controller to adjust the negative impedance value of the negative impedance circuit based on at least one signal parameter of the sympathetic nervous signal.

17. The sympathetic nervous signal sensing method of claim 15, further comprising:
filtering the fifth signal with a second filtering band using the post-processing circuit to output an electrocardiogram (ECG) signal; and
using a controller to adjust the negative impedance value of the negative impedance circuit based on at least one signal parameter of the electrocardiogram signal.
